(19) United States
(12) Patent Application Publication
(10) Pub. No.: US 2021/0161395 A1
(43) Pub. Date: Jun. 3, 2021

(54) NON-INVASIVE VENOUS WAVEFORM ANALYSIS FOR EVALUATING A SUBJECT

(71) Applicant: VANDERBILT UNIVERSITY, Nashville, TN (US)

(72) Inventors: Colleen M. BROPHY, Nashville, TN (US); Kyle M. HOCKING, Nashville, TN (US); Susan S. EAGLE, Nashville, TN (US); Franz J. BAUDENBACHER, Nashville, TN (US); Bret D. ALVIS, Nashville, TN (US)

(21) Appl. No.: 16/499,703

(22) PCT Filed: Apr. 13, 2018
(86) PCT No.: PCT/US2018/027439

§ 371 Date: Sep. 30, 2019

Related U.S. Application Data
(60) Provisional application No. 62/485,423, filed on Apr. 14, 2017.

(51) Int. Cl. A61B 5/0205; A61B 5/029; A61B 8/04; A61B 5/00

(52) U.S. Cl. CPC A61B 5/0205; A61B 5/029; A61B 8/04; A61B 5/7257; A61B 5/02133; A61B 5/4821; A61B 5/4845; A61B 2562/0247; A61B 2503/40; A61B 5/412

(57) ABSTRACT

An example method includes detecting, via a sensor, vibrations originating from a vein of a subject and obtaining an intensity spectrum of the detected vibrations over a range of frequencies. The method further includes using the obtained intensity spectrum to determine a metric selected from a group that includes: a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, and a volume responsiveness of the subject. An example computing device and an example non-transitory computer readable medium that are related to the method are disclosed as well.

### CROSS REFERENCE

This application claims priority to U.S. Provisional Patent Application Ser. No. 62/485423 filed Apr. 14, 2017, incorporated by reference herein in its entirety.

### STATEMENT REGARDING FEDERALLY SPONSORED RESEARCH OR DEVELOPMENT

This invention was made with government support under Contract Number 1549576 awarded by the National Science Foundation. The government has certain rights in the invention.

### BACKGROUND

Unless otherwise indicated herein, the materials described in this section are not prior art to the claims in this application and are not admitted to be prior art by inclusion in this section.

Acute decompensated heart failure is a common cause of patient hospitalization. Assessing a patient's pulmonary capillary wedge pressure (PCWP) is a useful tool for assessing vascular volume overload that can lead to such heart failure. PCWP assessment can also be used to assess the severity of heart failure and confirm the diagnosis of heart failure with preserved ejection fractions. When PCWP data is available, clinicians can prevent hospitalizations due to heart failure and can provide improvements in patient quality of life. Obtaining PCWP data is somewhat difficult because the procedure requires invasive placement of a pulmonary artery catheter, and, in some cases, the placement of an expensive invasive permanent device.

### SUMMARY

In one example, a method includes detecting, via a sensor, vibrations originating from a vein of a subject and obtaining an intensity spectrum of the detected vibrations over a range of frequencies. The method further includes using the obtained intensity spectrum to determine a metric selected from a group that includes: a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, and a volume responsiveness of the subject.

In another example, a computing device includes one or more processors, a sensor, and a computer readable medium storing instructions that, when executed by the one or more processors, cause the computing device to perform functions. The functions include detecting, via the sensor, vibrations originating from a vein of a subject and obtaining an intensity spectrum of the detected vibrations over a range of frequencies. The functions further include using the obtained intensity spectrum to determine a metric selected from a group that includes: a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, and a volume responsiveness of the subject.

In yet another example, a non-transitory computer readable medium stores instructions that, when executed by a computing device that includes a sensor, cause the computing device to perform functions. The functions include detecting, via the sensor, vibrations originating from a vein of a subject and obtaining an intensity spectrum of the detected vibrations over a range of frequencies. The functions further include using the obtained intensity spectrum to determine a metric selected from a group that includes: a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, and a volume responsiveness of the subject.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a schematic diagram of a computing device, according to an example embodiment.

FIG. 2 depicts a computing device, including a wireless sensor that is communicatively coupled to the computing device, according to an example embodiment.

FIG. 3A depicts a computing device, according to an example embodiment.

FIG. 3B depicts a sensor, according to an example embodiment.

FIG. 4A is a block diagram depicting a method, according to an example embodiment.

FIG. 4B depicts an intensity spectrum of vibrations originating from a subject's vein, according to an example embodiment.

FIG. 5 depicts a receiver operating curve for prediction of a subject's PCWP that is greater than 20 mmHg.

FIG. 6 depicts a correlation between subject NIVA score and subject volume status.

FIG. 7 depicts a correlation between subject NIVA score and subject volume status.

FIG. 8 depicts a correlation between PCWP and subject volume status.

FIG. 9 depicts a correlation between actual subject PCWP and subject PCWP determined based on subject NIVA score.

FIG. 10 depicts a correlation between subject cardiac output and subject volume status.

FIG. 11 depicts a correlation between actual change in subject cardiac output and change in subject cardiac output predicted based on subject NIVA score.

### DETAILED DESCRIPTION

As discussed above, direct measurement of PCWP has diagnostic value, but is inherently invasive and can be costly. Methods and systems for using non-invasive venous waveform analysis (NIVA) to indirectly determine PCWP and other subject metrics are disclosed herein.

PCWP is considered an important indicator for assessing the volume of blood within a subject's circulatory system at a particular time, also referred to herein as volume status. In addition to assessing volume status, NIVA can also be used to indirectly determine other useful subject metrics such as mean pulmonary arterial pressure, pulmonary artery diastolic pressure, left ventricular end diastolic pressure, left ventricular end diastolic volume, cardiac output, total blood volume, and volume responsiveness. These determined metrics may then be used to diagnose or treat various disorders that may afflict the subject.

More specifically, a sensor may be applied over a peripheral vein of a subject to detect vibrations caused by blood flow within the vein. A computing device may then obtain an intensity spectrum of the detected vibrations over a range of frequencies via signal processing. For instance, the computing device may perform a fast Fourier transform (FFT) upon a signal representing the detected vibrations to yield intensities corresponding to various respective vibration frequencies. The frequencies may represent the subject's respiratory rate, pulse rate, and various harmonics of the pulse rate. Next, the computing device may use the obtained intensity spectrum to determine a PCWP of the subject, or any other subject metric described herein. For example, the computing device (or a clinician) may determine the PCWP or other metric based on a known correlation between PCWP and the absolute intensities of the vibration frequencies and/or the relative intensity of one or more vibration frequencies compared to one or more other vibration frequencies.

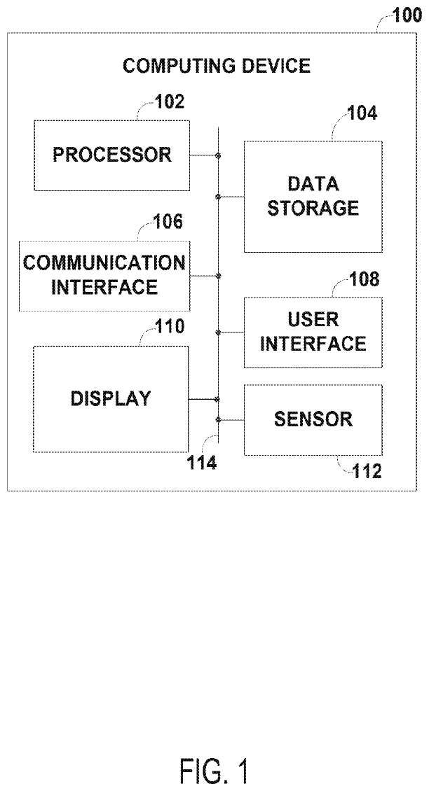

FIG. 1 is a simplified block diagram of an example computing device 100 that can perform various acts and/or functions, such as any of those described in this disclosure. The computing device 100 may be a mobile phone, a tablet computer, a laptop computer, a desktop computer, a wearable computing device (e.g., in the form of a wrist band), among other possibilities.

The computing device 100 includes one or more processors 102, a data storage unit 104, a communication interface 106, a user interface 108, a display 110, and a sensor 112. These components as well as other possible components can connect to each other (or to another device or system) via a connection mechanism 114, which represents a mechanism that facilitates communication between two or more devices or systems. As such, the connection mechanism 114 can be a simple mechanism, such as a cable or system bus, or a relatively complex mechanism, such as a packet-based communication network (e.g., the Internet). In some instances, a connection mechanism can include a non-tangible medium (e.g., where the connection is wireless).

The processor 102 may include a general-purpose processor (e.g., microprocessor) and/or a special-purpose processor (e.g., a digital signal processor (DSP)). In some instances, the computing device 100 may include more than one processor to perform functionality described herein.

The data storage unit 104 may include one or more volatile, non-volatile, removable, and/or non-removable storage components, such as magnetic, optical, or flash storage, and/or can be integrated in whole or in part with the processor 102. As such, the data storage unit 104 may take the form of a non-transitory computer-readable storage medium, having stored thereon program instructions (e.g., compiled or non-compiled program logic and/or machine code) that, when executed by the processor 102, cause the computing device 100 to perform one or more acts and/or functions, such as those described in this disclosure. Such program instructions can define and/or be part of a discrete software application. In some instances, the computing device 100 can execute program instructions in response to receiving an input, such as from the communication interface 106 and/or the user interface 108. The data storage unit 104 may also store other types of data, such as those types described in this disclosure.

The communication interface 106 can allow the computing device 100 to connect to and/or communicate with another other device or system according to one or more communication protocols. The communication interface 106 can be a wired interface, such as an Ethernet interface or a high-definition serial-digital-interface (HD-SDI). The communication interface 106 can additionally or alternatively include a wireless interface, such as a cellular or WI-FI interface. A connection provided by the communication interface 106 can be a direct connection or an indirect connection, the latter being a connection that passes through and/or traverses one or more entities, such as such as a router, switcher, or other network device. Likewise, a transmission to or from the communication interface 106 can be a direct transmission or an indirect transmission.

The user interface 108 can facilitate interaction between the computing device 100 and a user of the computing device 100, if applicable. As such, the user interface 108 can include input components such as a keyboard, a keypad, a mouse, a touch sensitive and/or presence sensitive pad or display, a microphone, a camera, and/or output components such as a display device (which, for example, can be combined with a touch sensitive and/or presence sensitive panel), a speaker, and/or a haptic feedback system. More generally, the user interface 108 can include any hardware and/or software components that facilitate interaction between the computing device 100 and the user of the computing device 100.

In a further aspect, the computing device 100 includes the display 110. The display 110 may be any type of graphic display. As such, the display 110 may vary in size, shape, and/or resolution. Further, the display 110 may be a color display or a monochrome display.

The sensor 112 may take the form of a piezoelectric sensor, a pressure sensor, a force sensor, an optical wavelength selective reflectance or absorbance measurement system, a tonometer, an ultrasound probe, a plethysmograph, or a pressure transducer. Other examples are possible. The sensor 112 may be configured to detect vibrations originating from a vein of a subject as further described herein.

As indicated above, the connection mechanism 114 may connect components of the computing device 100. The connection mechanism 114 is illustrated as a wired connection, but wireless connections may also be used in some implementations. For example, the communication mechanism 112 may be a wired serial bus such as a universal serial bus or a parallel bus. A wired connection may be a proprietary connection as well. Likewise, the communication mechanism 112 may also be a wireless connection using, e.g., Bluetooth® radio technology, communication protocols described in IEEE 802.11 (including any IEEE 802.11 revisions), cellular technology (such as GSM, CDMA, UMTS, EV-DO, WiMAX, or LTE), or Zigbee® technology, among other possibilities.

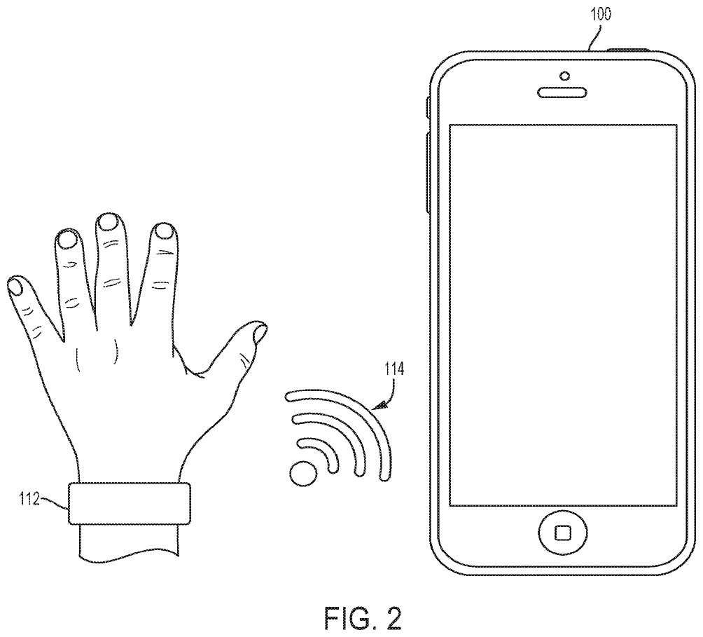

FIG. 2 depicts one embodiment of the computing device 100 and the sensor 112. In FIG. 2, the sensor 112 takes the faun of a wearable wristband that is worn by a human subject and the computing device 100 takes the form of a mobile phone. The sensor 112 may detect vibrations originating from a vein at the subject's wrist and wirelessly transmit (e.g., via Bluetooth®) a signal representing the detected vibrations. The computing device 100 may receive the signal for further processing as described further herein.

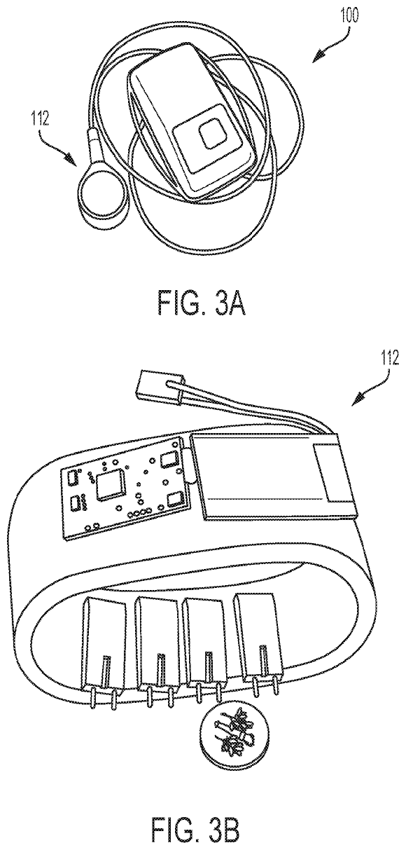

FIG. 3A depicts another embodiment of the computing device 100. In FIG. 3A, the computing device 100 is communicatively coupled to the sensor 112 via a wired connection.

FIG. 3B depicts an embodiment of the sensor 112, taking the form of a wristband.

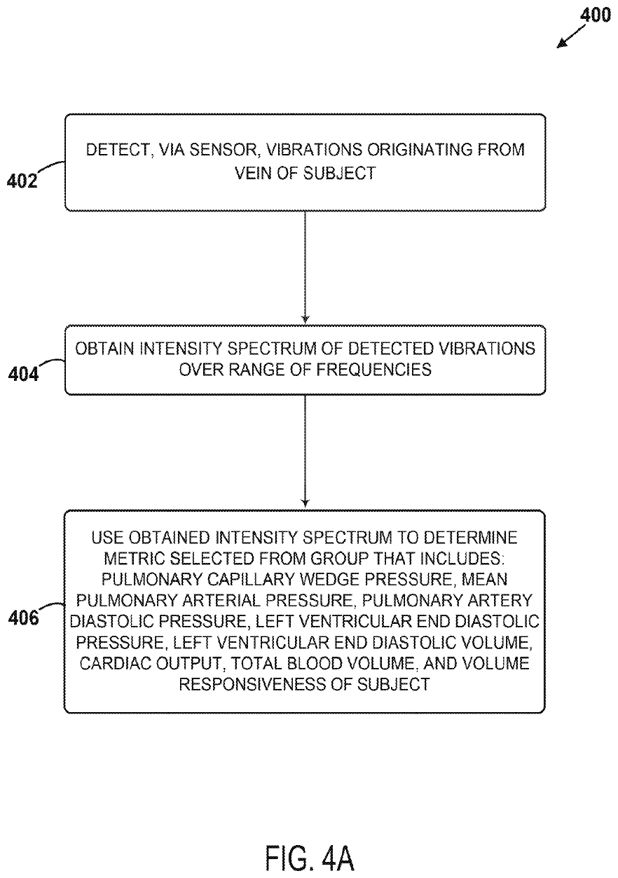

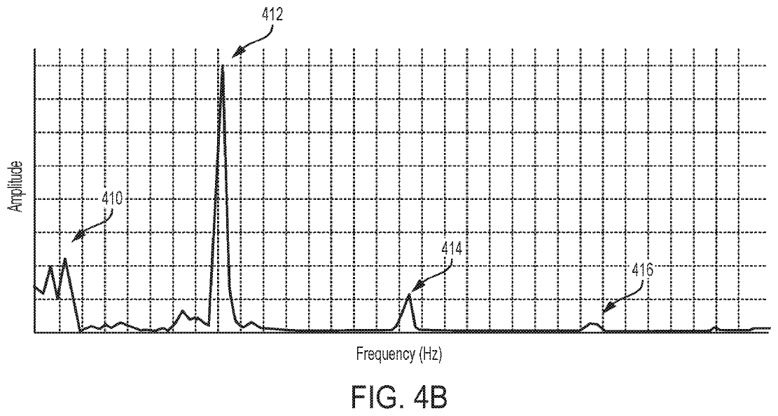

FIG. 4A is a block diagram of a method 400 that may be performed by andlor via the use of the computing device 100.

At block 402, the method includes detecting, via a sensor, vibrations originating from a vein of a subject. For example, the computing device 100, via the sensor 112, may detect vibrations originating from a vein (e.g., a vein wall) of a subject. In a specific example, the sensor 112 may be secured (e.g., via a Velcro strap) to the subject's skin above or near the subject's antebrachial vein. The sensor 112 may detect the vibrations caused by blood flow through the antebrachial vein (or another vein) as the vibrations are conducted through tissues such as the subject's skin. The subject may be human, but other animals are possible. As the sensor 112 detects the vibrations, the subject may be breathing spontaneously, e.g., without the aid of a mechanical ventilator, or with the aid of a mechanical ventilator.

At block 404, the method includes obtaining an intensity spectrum of the detected vibrations over a range of frequencies (e.g., 0.05 Hz-25 Hz). More specifically, the computing device 100 may perform a fast Fourier transform (FFT) upon a signal representing the detected vibrations that is received from the sensor 112. Performing the FFT may yield one or more intensities corresponding respectively to one or more frequencies of the detected vibrations. Frequencies of interest such as a subject's respiratory rate, a pulse rate, and harmonics or multiples of the pulse rate may take the form of “peaks” within the obtained intensity spectrum. Such peaks may take the form of local (or global) maxima of signal intensity with respect to signal frequency. The FFT may be non-linear or any other foul) of FFT. In some examples, the computing device 100 may perform the FFT after the computing device 100 performs an autocorrelation operation, a Hilbert-Huang Transform (HHT), or an empirical mode decomposition (EMD) upon the signal representing the vibrations.

FIG. 4B is a graphical depiction of an arbitrary intensity spectrum yielded by performing an FFT on a signal representing vibrations that are detected from a vein wall. The arbitrary intensity spectrum represents intensities of vein vibrations corresponding to various respective frequencies. FIG. 4B shows intensity or amplitude peaks 410, 412, 414, and 416 that may represent frequencies of interest for establishing correlations between vein vibration data and various subject metrics discussed below.

At block 406, the method includes using the obtained intensity spectrum to determine a metric selected from a group that includes: a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, and a volume responsiveness of the subject. More specifically, the computing device 100 or a user may use the obtained intensity spectrum to determine one or more of the aforementioned subject metrics.

This process may involve using known statistical correlations between previously collected intensity spectra of subject vein vibrations and the aforementioned subject metrics. For example, vein vibration data may be collected for a number of subjects while one or more of the aforementioned metrics are directly measured for each of the subjects. This data may then be used to determine statistical correlations between the collected vein vibration data and the aforementioned subject metric data. More specifically, such correlations between the vein vibration data and the subject metric data can be approximated as mathematical functions using various statistical analysis or “curve fitting” techniques (e.g., least squares analysis). As such, future subject metrics may be determined indirectly (e.g., without direct measurement) and non-invasively with the sensor 112 by performing the identified mathematical functions upon subsequently collected vein vibration intensity data.

In a specific example, PCWP may be determined by using the following derived formula: NIVA score=6.5+4.8(0.92A₀+2A₁+0.4A₂+0.2A₃)/(A₀+A₁+A₂+A₃)+44*(A₄+A₅+A₆+A₇+A₈)/(A₁+A₂+A₃+A₄+A₅+A₆+A₇+A₈)+0.0296(A₀/A₁). In some examples, the determined NIVA score is equal to a value predicted to be equal to the subject's PCWP. In this example, A₀ is an intensity of the subject's respiration rate, A₁ is an intensity of the subject's pulse rate (f₁), and A₂, A₃, A₄, A₅, A₆, A₇, and A₈ are respective intensities of 2f₁, 3f₁, 4f₁, 5f₁, 6f₁, 7f₁, and 8f₁. The respiration rate, pulse rate, and harmonics of the pulse rate may be identified as frequencies at which local or global maxima of intensity occur.

The determined PCWP or other determined subject metric may be used to diagnose or treat one or more of the following disorders: hypervolemia, hypovolemia, euvolemia, dehydration, heart failure, tissue hypoperfusion, myocardial infarction, hypotension, valvular heart disease, congenital heart disease, cardiomyopathy, pulmonary disease, arrhythmia, drug effects, hemorrhage, systemic inflammatory response syndrome, infectious disease, sepsis, electrolyte imbalance, acidosis, renal failure, hepatic failure, cerebral injury, thermal injury, cardiac tamponade, preeclampsia/eclampsia, or toxicity. The determined PCWP or other determined subject metric may also be used to diagnose respiratory distress or hypoventilation due to one or more of the following conditions: pneumonia, cardiac disorders, sepsis, asthma, obstructive sleep apnea, hypopnea, anesthesia, pain, or narcotic use.

The method 400 may be performed to diagnose or treat a subject that is suffering from increased or decreased cardiac output compared to control or increased or decreased intravascular volume status compared to control. The method 400 may also be performed for subjects that are to undergo cardiac catheterization or have undergone cardiac catheterization.

The determined PCWP or other determined subject metric may additionally be used to determine whether intravenously administering a fluid to the subject would increase, decrease, or not significantly affect a cardiac output of the subject.

In some examples, the method 400 may be performed a first time prior to treatment or diagnosis of one or more disorders and a second time after carrying out the treatment or determining the diagnosis.

The method 400 may involve iterative derivation using leverage plots of the contribution of one or more of f₀-f₈ to the data collected for pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, or volume responsiveness. The log worth of the values may be used to determine optimal weighting factors and constants to define NIVA volume index or score. In this case, the algorithm may be a ratio of a sum of the higher harmonics of pulse rate to a sum of the amplitude of lower harmonics of pulse rate modified by a constant that normalizes the data to a known clinical output such as a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, and a volume responsiveness of the subject according to a(f₀)+b(f₁)+c(f₂)+d(f₃)+e(f₄)+(f₅)+h(f₆)+i(f₇)+j(f₈)+(κ) divided by l(f₀)+m(f₁)+n(f₂)+o(f₃)+p(f₄)+q(f₅)+r(f₆)+s(f₇)+t(f₈)+(λ), where f₀-f₈ are the frequencies derived from a fast Fourier transformation of the venous waveform and κ, λ, a, b, c, d, e, g, h, i, j, 1, m, n, o, p, q, r, s, t are numerical constants that weight and normalize the algorithm.

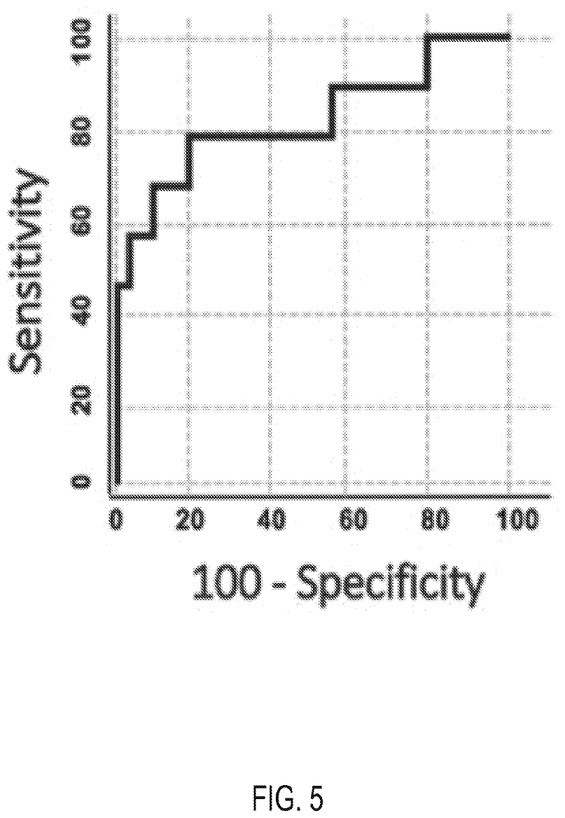

FIG. 5 depicts a ROC curve comparing vein vibration data to PCWP data. An area under the curve is 0.805, demonstrating the successful use of the method 400 to detect a PCWP above 20 mmHg. Patients who have a PCWP greater than 20 mmHg are not expected to be volume responsive and have an increased intravascular volume status.

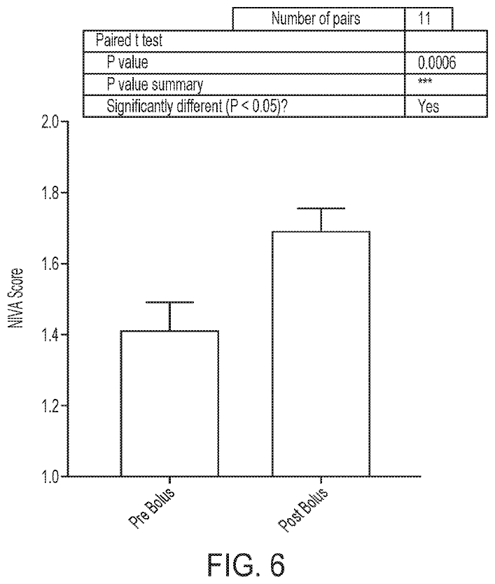

FIG. 6 depicts a correlation between subject NIVA score and subject volume status. As shown, NIVA score is shown to increase upon the administration of fluids (e.g., a bolus) and the resultant increased intravascular volume.

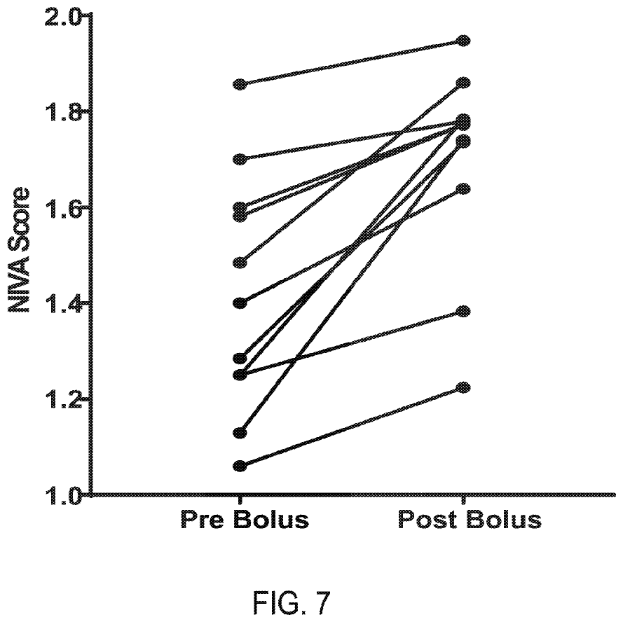

FIG. 7 depicts raw data showing the correlation between subject NIVA score and subject volume status. Eleven patients who had invasive right heart catheterization also had a NIVA measurement taken on them before and after administration of 500 mL of crystalloid. There was a significant (p<0.05) increase in NIVA score with the administration of fluids.

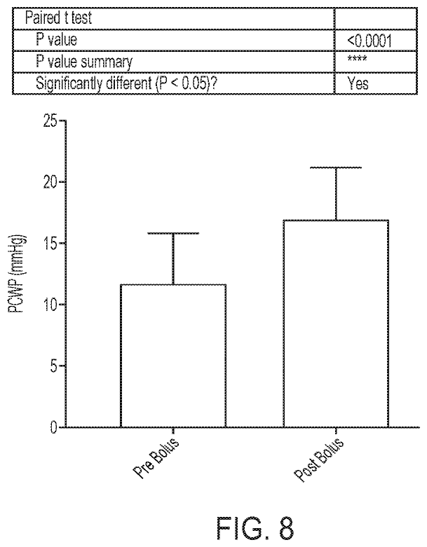

FIG. 8 depicts a correlation between PCWP and subject volume status. As shown, PCWP is shown to increase upon the administration of fluids and the resultant increased intravascular volume. NIVA score and PCWP significantly increased by 21.4% (p=0.006) and 33.3% (p<0.001), respectively, after fluid administration.

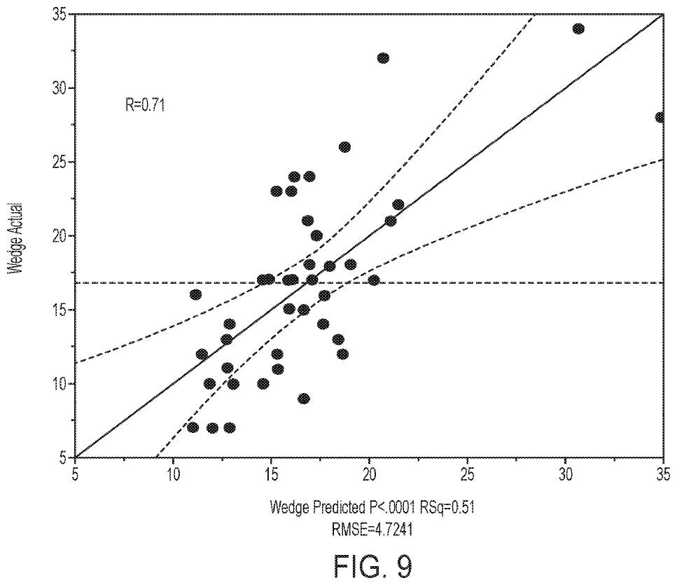

FIG. 9 depicts a correlation between actual subject PCWP and subject PCWP determined based on subject NIVA score. Forty nine patients that had invasive right heart catheterization were equipped with a NIVA device. These patients had PCWP measured which correlated with the NIVA measurement (p<0.05, R=0.71).

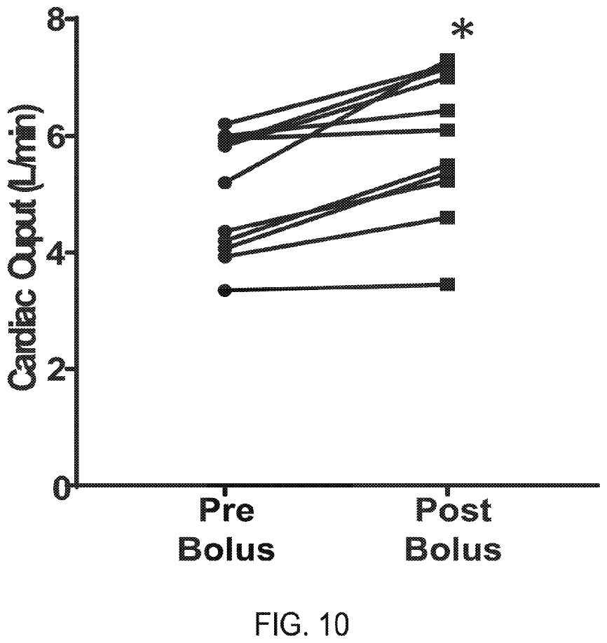

FIG. 10 depicts a correlation between subject cardiac output and subject volume status. Thirteen patients who had invasive right heart catheterization underwent a fluid administration where cardiac output was measured before and after a 500 mL fluid bolus.

There was a significant (p<0.05) increase in in cardiac output with the administration of fluids.

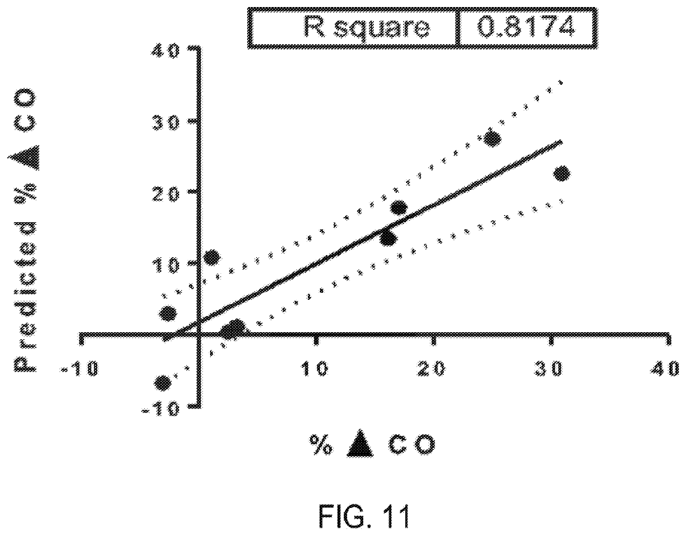

FIG. 11 depicts a correlation between actual change in subject cardiac output and change in subject cardiac output predicted based on subject NIVA score. Predicted change in cardiac output (N=9) correlated strongly with thermodilution-based cardiac output measurements with r²=0.82.

The following includes further details related to the methods and systems described above.

### EXAMPLE 1

### Clinical Study of Non-Invasive Venous Waveform Analysis (NIVA) for Prediction of a High Pulmonary Capillary Wedge Pressure

Acute decompensated heart failure is the leading cause of hospitalization in patients over the age of 65. Pulmonary capillary wedge pressures (PCWP) have been considered the gold standard for assessing volume overload. PCWP have also been used to gauge the severity of heart failure and confirm the diagnosis of heart failure with preserved ejection fractions. When continuous pulmonary artery pressure readings are available to clinicians, a reduction in heart failure hospitalizations and an improvement in quality of life have been demonstrated. Limitations to pulmonary capillary wedge pressures are that they require an invasive placement of a pulmonary artery catheter, and, in some cases, the placement of an expensive invasive permanent device. We hypothesize that non-invasive venous waveform analysis (NIVA) that utilizes piezoelectric sensors to detect vascular harmonics can predict high (>20 mmHg) pulmonary capillary wedge pressures without the need for an invasive procedure.

Methods:

Patients (n=43) undergoing cardiac catheterization were enrolled in this Vanderbilt University Institutional Review Board approved protocol. Prior to the patient undergoing their cardiac catheterization, the NIVA device was placed over the median antebrachial vein. Over the course of the procedure, continuous, non-invasive, real-time data of the vascular harmonics were obtained. Upon completion of the procedure, the piezoelectric sensors were removed from the patient and the data were imported into LabChart software (ADInstruments, Colorado Springs, Colo., USA). The data were transformed into the frequency domain using Fourier transformations to display the patient signal as a function of sine waves and their corresponding power. The peaks corresponding to the patients' heart rate (f₁-f₈) were measured as a function of power and inputted into our “NIVA signal” algorithm (see description above relating to at least block 406 of the method 400). The PCWP was obtained from the pulmonary artery catheter used during the cardiac catheterization, per routine. To determine NIVA signal's ability to predict an elevated PCWP (above 20 mmHg) a receiver operator characteristic (ROC) curve was used.

Results:

The ROC curve comparing the NIVA signal against the PCWP revealed an area under the curve of 0.805, demonstrating NIVA's ability to detect a wedge pressure above 20 mmHg (See FIG. 5).

Conclusion:

In patients undergoing cardiac catheterizations, a patient's NIVA signal was able to detect high pulmonary capillary wedge pressures. This non-invasive method can provide a real-time assessment of a patient's cardiac condition by informing a clinician when the pulmonary capillary wedge pressure is high.

### EXAMPLE 2

### Clinical Study of Non-Invasive Venous Waveform Analysis (NIVA) for Prediction of Fluid Responsiveness in Spontaneously Breathing Subjects

In this study, we evaluated the correlation of Non-invasive venous waveform analysis (NIVA) with fluid responsiveness, as defined by the change in cardiac output in response to a crystalloid fluid bolus.

Methods

Eleven patients undergoing elective right heart catheterization were included in this study that was approved by the Vanderbilt University Medical Center Institutional Review Board. Mechanically ventilated patients were excluded. NIVA sensors were applied over median antebrachial vein and data was collected immediately pre- and post-infusion of a 500-mL bolus of crystalloid solution. Pulmonary capillary wedge pressure (PCWP) and, if available, cardiac output (CO) was also recorded pre- and post-infusion. NIVA score was calculated using a linear regression model with covariates including the 1^{st} through 4^{th} harmonics of pulse rate. Predicted change in cardiac output was calculated as a simple linear model including the calculated NIVA score and a regression coefficient. Data were analyzed using paired Student's t-tests.

Results

Pre- to post-bolus NIVA score and PCWP were significantly increased by 21.4% (p=0.006) and 33.3% (p<0.001), respectively. See FIGS. 6 and 8. Predicted change in cardiac output (N=9) correlated strongly with thermodilution-based cardiac output measurements with r²=0.82. See FIG. 11.

Conclusions

In spontaneously breathing patients undergoing right heart catheterization, NIVA correlated strongly with changes in cardiac output as measured by thermodilution. NIVA is a promising non-invasive modality for measurement of fluid responsiveness in spontaneously breathing individuals.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

1. A method comprising:
(a) detecting, via a sensor, vibrations originating from a vein of a subject;
(b) obtaining an intensity spectrum of the detected vibrations over a range of frequencies; and
(c) using the obtained intensity spectrum to determine a metric selected from a group comprising: a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, and a volume responsiveness of the subject.

2. The method of claim 1, wherein the sensor comprises a piezoelectric sensor, a pressure sensor, a force sensor, an optical wavelength selective reflectance or absorbance measurement system, a tonometer, an ultrasound probe, a plethysmograph, or a pressure transducer.

3. The method of claim 1, wherein the vibrations comprise vibrations of a wall of the vein produced by fluid flowing through the vein.

4. The method of claim 1, wherein the sensor is positioned proximately to a peripheral vein of the subject, and wherein the vibrations originate from the peripheral vein of the subject.

5. The method of claim 1, wherein the subject is a human subject or an animal subject.

6. The method of claim 1, wherein the subject is breathing spontaneously while the vibrations are detected.

7. The method of claim 1, wherein the range of frequencies is 0.05 Hz to 25 Hz.

8. The method of claim 1, wherein obtaining the intensity spectrum comprises performing a fast Fourier transform (FFT) upon a signal representing the detected vibrations to yield one or more intensities corresponding respectively to one or more frequencies of the detected vibrations.

9. The method of claim 8, wherein performing the FFT comprises performing the FFT after performing an autocorrelation of the signal.

10. The method of claim 8, wherein performing the FFT comprises performing the FFT after performing a Hilbert-Huang Transform (HHT) or an empirical mode decomposition (EMD) upon the signal.

11. The method of claim 8, wherein performing the FFT comprises performing a nonlinear FFT.

12. The method of claim 8, wherein using the obtained intensity spectrum comprises calculating a weighted sum of one or more intensities yielded by the FFT.

13. The method of claim 12, wherein calculating the weighted sum comprises calculating a weighted sum of respective intensities of the subject's respiration rate, pulse rate, and one or more harmonics of the pulse rate.

14. The method of claim 13, wherein using the obtained intensity spectrum further comprises dividing the weighted sum by a sum of the respective intensities of the respiration rate, the pulse rate, and the one or more harmonics of the pulse rate.

15. The method of claim 8, wherein using the obtained intensity spectrum comprises calculating a second sum of respective intensities of two or more harmonics of a pulse rate of the subject.

16. The method of claim 15, wherein using the obtained intensity spectrum further comprises dividing the second sum by a sum of respective intensities of the subject's pulse rate and one or more harmonics of the pulse rate.

17. The method of claim 8, wherein using the obtained intensity spectrum comprises calculating a quotient of an intensity of the respiration rate divided by an intensity of the pulse rate.

18. The method of claim 1, wherein A₀ is an intensity of the subject's respiration rate, A₁ is an intensity of the subject's pulse rate (f₁), A₂, A₃, A₄, A₅, A₆, A₇, and A₈ are respective intensities of 2f₁, 3f₁, 4f₁, 5f₁, 6f₁, 7f₁, and 8f₁, and wherein using the obtained intensity spectrum comprises calculating a score equal to: 6.5+4.8(0.92A₀+2A₁+0.4A₂+0.2A₃)/(A₀+A₁+A₂+A₃)+44*(A₄+A₅+A₆+A₇+A₈)/(A₁+A₂+A₃+A₄+A₅+A₆+A₇+A₈)+0.0296(A₀/A₁).

19. The method of claim 1, wherein using the obtained intensity spectrum comprises using an algorithm to generate a numerical score.

20. The method of claim 1, further comprising iterative derivation using leverage plots of the contribution of one or more of f₀ (respiration rate), f₁ (pulse rate), 2f₁, 3f₁, 4f₁, 5f₁, 6f₁, 7f₁, and/or 8f₁ to the data collected for pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, or volume responsiveness, wherein log worth of the values are used to determine optimal weighting factors and constants to define NIVA volume index or score, wherein the algorithm comprises calculating a ratio of a sum of the higher harmonics of pulse rate to a sum of the amplitude of lower harmonics of pulse rate modified by a constant that normalizes the data to a known clinical output such as a pulmonary capillary wedge pressure (PCWP), a mean pulmonary arterial pressure, a pulmonary artery diastolic pressure, a left ventricular end diastolic pressure, a left ventricular end diastolic volume, a cardiac output, total blood volume, or a volume responsiveness of the subject according to a(f₀)+b(f₁)+c(f₂) d(f₃)+e(f₄)+g(f₅)+h(f₆)+i(f₇)+j(f₈)+(κ) divided by l(f₀)+m(f₁)+n(f₂)+o(f₃)+p(f₄)+q(f₅)+r(f₆)+s(f₇)+t(f₈)+(λ), wherein f₀ and f₁ are frequencies derived from a fast Fourier transformation of the venous waveform and κ, λ, a, b, c, d, e, g, h, i, j, l, m, n, o, p, q, r, s, t are numerical constants that weight and normalize the algorithm.

21. The method of claim 1, further comprising using the determined metric to diagnose one or more of the following disorders:
hypervolemia, hypovolemia, euvolemia, dehydration, heart failure, tissue hypoperfusion, myocardial infarction, hypotension, valvular heart disease, congenital heart disease, cardiomyopathy, pulmonary disease, arrhythmia, drug effects, hemorrhage, systemic inflammatory response syndrome, infectious disease, sepsis, electrolyte imbalance, acidosis, renal failure, hepatic failure, cerebral injury, thermal injury, cardiac tamponade, preeclampsia/eclampsia, or toxicity.

22. The method of claim 21, wherein the method comprises carrying out steps (a)-(c) a first time prior to treatment of the one or more disorders and a second time after carrying out the treatment.

23. The method of claim 1, wherein the subject is suffering from increased or decreased cardiac output compared to control or increased or decreased intravascular volume status compared to control.

24. The method of claim 1, wherein the subject is to undergo cardiac catheterization, or has undergone cardiac catheterization or a minimally or non-invasive method to determine cardiac output or volume status.

25. The method of claim 1, further comprising determining an effect administering a fluid to the subject would have on a cardiac output of the subject.

26. The method of claim 1, further comprising: performing steps (a)-(c) to diagnose respiratory distress or hypoventilation due to one or more of the following conditions: pneumonia, cardiac disorders, sepsis, asthma, obstructive sleep apnea, hypopnea, anesthesia, pain, or narcotic use.

27. The method of claim 1, wherein using the obtained intensity spectrum comprises using the obtained intensity spectrum to determine a PCWP of the subject.

28. A computing device comprising:
one or more processors;
a sensor; and
a computer readable medium storing instructions that, when executed by the one or more processors, cause the computing device to perform the method of claim 1.

29. A non-transitory computer readable medium storing instructions that, when executed by a computing device, cause the computing device to perform the method of claim 1.
